(19) United States
(12) Patent Application Publication
(10) Pub. No.: US 2026/0232184 A1
(43) Pub. Date: Aug. 13, 2026

(54) Oculometry Apparatus Having a 2-D Display Projector and Retroreflective Screen

(71) Applicant: NATUS MEDICAL INCORPORATED, Middleton, WI (US)

(72) Inventors: Yan Zhou, Pleasanton, CA (US); John Allison, Pleasanton, CA (US)

(73) Assignee: Natus Acquisition II, LLC, Schaumburg, IL (US)

(21) Appl. No.: 19/155,448

(22) PCT Filed: Feb. 14, 2024
(86) PCT No.: PCT/US2024/015771

§ 371 Date: Aug. 11, 2025

Related U.S. Application Data
(60) Provisional application No. 63/484,877, filed on Feb. 14, 2023.

(51) Int. Cl. A61B 3/113; A61B 3/00; A61B 3/14

(52) U.S. Cl. CPC A61B 3/113; A61B 3/0008; A61B 3/005; A61B 3/14

(57) ABSTRACT

An oculometry apparatus including a goggle device comprising a projection device that includes a microLED display device comprising a light-emitting pixel array and an optical lens positioned in optical communication with the microLED display device and configured to project light within a field of view of the optical lens. The apparatus further includes a retroreflective screen positioned to overlap the field of view of the optical lens. The projection device may be positioned proximate to one or more patient eyes and the retroreflective screen may retro-reflect light in the direction of the one or more patient eyes.

### RELATED APPLICATIONS

This application is a national phase application of and claims priority under 35 U.S.C. § 371 of PCT Patent Application Serial No. PCT/US24/15771 (Attorney Docket No. 9225.00353) filed on Feb. 14, 2024 and titled Oculometry Apparatus Having a2-D Display Projector and Retroreflective Screen, which in turn is a PCT application of and claims priority under 35 U.S.C. § 120 of U.S. Patent Application Ser. No. 63/484,877 (Attorney Docket No. 4735.01135) filed on Feb. 14, 2023 and titled Oculometry Apparatus Having a 2-D Display Projector and Retroreflective Screen. The contents of these applications are incorporated herein by reference.

### FIELD OF THE INVENTION

The present invention relates to systems and methods for oculometry examination, including videonystagmography (VNG).

### BACKGROUND

Current oculometry apparatuses have a number of disadvantages. One problem is the mechanical moving nature of laser-driving ocular motors necessary in laser-based solutions which can have long-term reliability issues. There is a need in the art for a solution achieving the same function in terms of effectively lighting up a stationary stimuli spot or moving a stimuli spot on a screen in both the horizontal direction and the vertical direction using only solid-state devices.

Another problem in the art is the need of a separate stationary laser in current VNG goggle solutions. Such solutions limit the ability to use retroreflective screens, thereby increasing the necessary brightness of light for performing the oculometric examination. There is a need in the art for a solution that is sufficiently lightweight and compact that it can be positioned within a goggle device.

Another problem in the art is single stimuli spot limitations resulting from the use of a single movable laser beam having limited moving speed because the ocular motor cannot be driven fast enough to produce a real time raster-scan movie. There is a need in the art for a solution to generate a real-time 2D pattern in the form of a video/movie that can be produced to enable additional balance related tests like the optokinetic test, which current ocular motor-based laser spot solutions cannot provide and hence require a separate display screen will be needed.

Another problem in the art is the size and weight of the ocular motor sub-assemblies, because the overall weight and size of goggle-based oculometry solutions is a key usability factor. Current solutions are unduly large and heavy so as to preclude their usefulness. There is a need in the art for lighter-weight and more compact oculometry solutions that facilitate being placed within a goggle of desirably small size and comfortable weight to improve usefulness, particularly in comparison to that of the ocular motor subassembly.

Another problem in the art is related to the relatively complicated mechanical design of the ocular motor sub-assembly and the associated difficulty in terms of manufacturability in comparison to a very simple modular design that can be fabricated separately and easily integrated into an oculometry goggle. Current ocular motors are a two-shaft step motor that is combined with a bevel gearing system to drive the laser to change its pointing direction in both the x and y directions. To avoid or substantially reduce the mechanical coupling of the x and y movement, current designs are becoming more and more complicated with ball bearings, high precision bevel gears, precision adjustment of the two bevel gears' relative positions using shims, and the like. The two-shaft motor is directly mounted on a PCBA board which makes manufacturing difficult and not modular. There is a need for a sub-assembly that can be manufactured separately from a goggle housing, independently tested for quality control, and then easily integrated into a goggle oculometry device.

Another problem in the art is related to the cost of the ocular motor subassembly. With augmented reality (AR) and virtual reality (VR) goggles being a significant ongoing development stream, improved self-light emitting micro-displays in terms of cost, size and display brightness are being commercialized. Therefore, a solution utilizing such devices will be advantageous.

Another problem in the art is related to power consumption and hence brightness of the stimuli or video scene on a viewing surface. Due to the fact that many oculometry devices are designed to be driven by a USB 3.0 cable with limited power supply, electrical power is limited. Hence, when power delivered by the USB cable is converted to optical power, the optical power is also limited. As a result, when a two-dimensional display projector is used, the brightness of the display on a screen or wall about 1.5 to 2 meters away is generally limited to such an extent that it is not clearly visible by a patient in a room with room lighting on. This is especially true when a short throw ratio optical projector is required as in the case of a VNG goggle that requires an angular field of view of at least ±30 degrees in the horizontal direction and ±25 degrees in the vertical direction. Accordingly, there is a need for a solution that can utilize a retroreflective screen that will direct light rays striking the screen to mostly retroreflectively return along and around its incident light path, so by arranging the light exit port of the two-dimensional display optical projector somewhere in between the two eyes of a patient with a certain design, much more optical energy can be directed from retroreflective screen back to the two patient eyes when compared with that of a wall from which an incident beam will be scattered/reflected uncontrollably with much less percentage of light being sent back to the patient eyes.

Another problem in the art is related to the projection angular field of view (FOV). Current solutions have optical projectors that either have very large size with short throw ratios or limited angular FOV not sufficient for oculometry, specifically videonystagmography (VNG) applications if the projector is sufficiently compact to be integratable inside the oculometry goggle. There is a need in the art for an oculometry solution with a wide-angle projection lens conventionally and typically used in wide angle imaging applications. Due to the fact that for VNG stimuli projection applications, there is no need to have the projected stimuli spot having extremely well-controlled size across the full FOV range, as long as the subtended angular range of the stimuli spot is less than one degree per the standard S3.45-2009_R2019, optical magnification difference may produce a stimuli spot size difference that can be well controlled to meet the one degree subtended angular range requirement and optical distortion can be calibrated such that for a particular angle or stimuli spot position, a corresponding pixel or binned group of pixels can be turned on.

### BRIEF DESCRIPTION OF THE DRAWINGS

FIG. 1 is a perspective view of an oculometry apparatus according to an embodiment of the invention.

FIG. 2 is a schematic representation of the oculometry apparatus of FIG. 1.

FIG. 3 is a representative depiction of a pixel array of a display device of a projection device according to an embodiment of the invention.

FIG. 4 is a perspective view of a goggle device of the oculometry apparatus of FIG. 1.

FIG. 5 is a flowchart illustrating a method of performing an oculometric examination according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

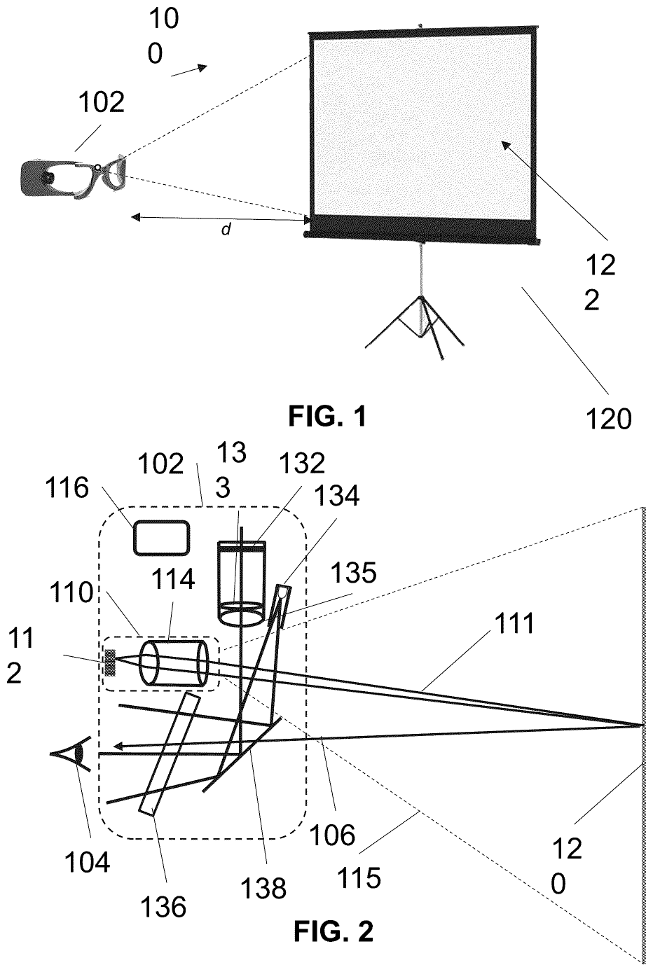

An embodiment of the invention, as shown and described by the various figures and accompanying text, provides an oculometry apparatus that may be used for performing videonystagmography. Referring now to FIG. 1, an apparatus 100 according to an embodiment of the invention is presented. The apparatus 100 may comprise a goggle device 102 and a retroflective screen 120. The goggle device 102 may be configured to be worn by a patient. The goggle device 102 may comprise a projection device 110 as shown in FIG. 2. The projection device 110 may be operable to emit light onto the retroreflective screen 120 configured to elicit a biological response in a patient observing the retroreflective screen 120 for vestibular assessment, for example, nystagmus. The unique reflective property of the retroreflective screen 120, reflecting light projected thereon back in the direction from which it was projected, as compared to a standard screen that reflects light projected thereon with greater scattering and/or only reflects back in the direction of projection if the angle of incidence is very small or zero, results in the necessary intensity of light emitted by the projection device being substantially lower. This may facilitate a smaller and lighter-weight display device that may be positioned in a greater variety of positions. Such positioned may include within the goggle device 102, as shown in the present embodiment. It is contemplated and included within the scope of the invention that the display device may be positioned in other locations, including, but not limited to, on headgear attached to or positioned adjacent a patient head, on a surface adjacent the patient head, or the like.

The projection device 110 may comprise a display device 112. The display device 112 may be operable to emit light in a grid configuration. In some embodiments, the display device 112 may be a microLED display device and comprising a light-emitting pixel array. Such an embodiment may facilitate the display of displaying light to cover a two-dimensional space, namely, a retroreflective viewing surface 122 of the retroreflective screen 120.

The display device 112 may comprise one or more of the following features. In some embodiments, the display device may consume three watts or less. In some embodiments, the display device may consume two watts or less. In some embodiments, the display device may consume one watt or less. In some embodiments, the display device may consumer 0.5 watts or less. In some embodiments, the display device may consumer 0.4 watts or less. In some embodiments, the display device may consumer 0.3 watts or less. The display device 112 may be configured to emit lighting within the visible spectrum, namely, light having a peak wavelength within a range from 380 nanometers (nm) to 750 nm. In some embodiments, the display device 112 may be configured to emit light that is monochromatic, i.e. having a narrow wavelength band. In some embodiments, the display device 112 may be configured to emit a green monochromatic light having a peak wavelength within a range from 495 nm to 570 nm.

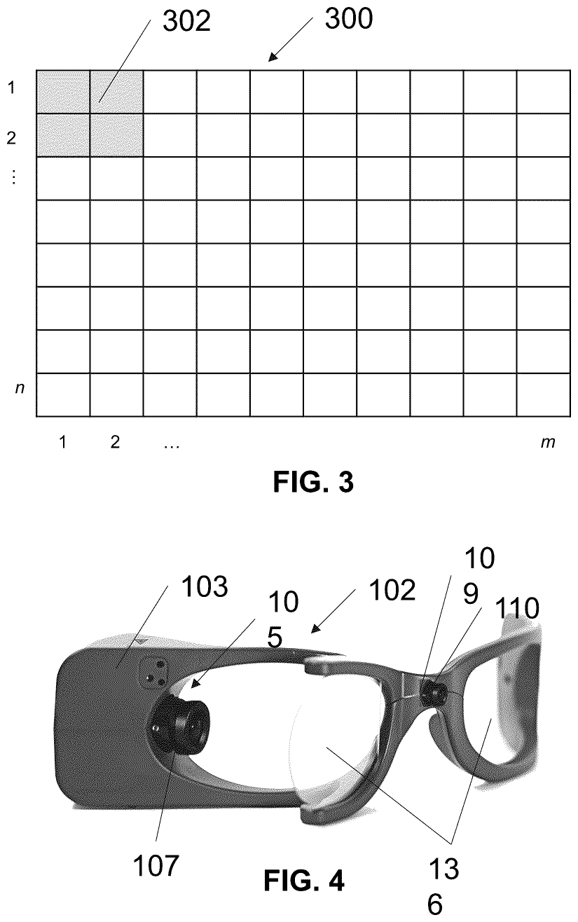

The pixel array may be defined by a horizontal pixel count m and a vertical pixel count n, as shown by array 300 in FIG. 3. In some embodiments, the horizontal pixel count may be at least 120 pixels. In some embodiments, the vertical pixel count may be at least 100 pixels. In some embodiments, the horizontal pixel count may be at least 480 pixels and the vertical pixel count may be at least 400 pixels. It is further contemplated that the horizontal to vertical pixel ratio may be 6:5, and in further embodiments, any array, cropped or non-cropped, having such a ratio of 6:5, a horizontal pixel count of at least 120 pixels, and a vertical pixel count of at least 100 pixels.

In some embodiments, the pixel array 300 may be operable such that groups of adjacent may be “binned” and operated concurrently, acting as a single pixel, as shown by pixel subgroup 302. For example, where the horizontal pixel count is at least 480 pixels and the vertical pixel count is at least 400 pixels, groups of four pixels, in a 2×2 array, may be binned and operated concurrently as a single pixel. As another example, where the horizontal pixel count is at least 960 pixels and the vertical pixel count is at least 800 pixels, groups of sixteen pixels, in a 4×4 array, may be binned and operated concurrently as a single pixel. It is contemplated and included within the scope of the invention that any number of pixels in any arrangement may be binned, such that the effective array of the binned pixels is at least 120 horizontal binned pixels by 100 vertical binned pixels.

To minimize the weight of a power supply element 116, for example, a battery or a power transformer or an adaptor, in terms of voltage or current regulation, of the goggle device 102, power consumption by the display device 112 should be minimized. In the present embodiment, the maximum power draw of the display device 112 is less than or equal to 15 mW. Additionally, the optical power of light emitted by the display device 112 must be sufficiently bright such that its reflection from the display screen 120 is visible by a patient eye 104 at a distance d, as discussed in greater detail below. In some embodiments, the optical power of light emitted from each pixel or each binned pixel of the pixel array may be at least 16 nW, at least 64 nW, at least 256 nW, or within a range from at least 8 nW to at least 512 nW.

Additionally, the display device 112 may be operable to change or light up the pixels and/or binned pixels sufficiently fast, either simultaneously or sequentially, to simulate motion, generating a video display. Each change of the display device 112 may be considered a frame of the video display, with a resulting number of frames per second (FPS) depending on how quickly the display device 112 can change the pixels/binned pixels. In some embodiments, the display device 112 may have a frame rate of at least 15 FPS. In some embodiments, the display device 112 may have a frame rate of at least 20 FPS. In some embodiments, the display device 112 may have a frame rate of at least 25 FPS. In some embodiments, the display device 112 may have a frame rate of at least 30 FPS. In some embodiments, the display device 112 may have a frame rate of at least 35 FPS. In some embodiments, the display device 112 may have a frame rate of at least 40 FPS.

The projection device 110 may further comprise an optical lens 114 positioned in optical communication with the display device 112. Specifically, the optical lens 114 may be positioned in an illumination path 111 of the projection device 110 defined by a direction of light emission of the display device 112. Such direction of emission may start with an emitting lens (not shown) fabricated on top of each microLED pixel of the display device 112. Light emitted from the display device 112 may be refracted by the optical lens 114 and projected therefrom. The directions of light projected from the optical lens 114 may be defined as a field of view 115 of the optical lens 114/projection device 110. The retroreflective screen 120 may be positioned relative to the goggle device 102 such that it overlaps the field of view 115. In the present embodiment, the optical lens 114 defines a field of view 115 in front of the projection device 110 characterized by a 60° horizontal angular dimension and a 50° vertical angular dimension. In another embodiment, the field of view 115 may be characterized by a horizontal angular dimension within a range from 50° to 70° and a vertical angular dimension within a range from 40° to 60°.

The goggle device 102 may further comprise a plurality of components to enable eye imaging. The components may include an infrared light source 134, an imaging sensor 132, a dichroic reflective device 138, and a bandpass filter and/or electrooptic device 136. The dichroic reflective device 138 and the bandpass filter and/or electrooptic device 136 can be combined into one device shown as 136 in FIG. 4. The infrared light source 134 may be positioned to emit infrared light, i.e. light having a peak intensity within a wavelength range from 700 nm to 1,000 nm, that is incident upon the patient eye 104. Such incidence may result from light emitted by the infrared light source 134 being directly incident on the patient eye 104 or indirectly incident after being reflected by a reflective structure, such as a hot mirror 138 that may also be comprised by the goggle device 102.

The patient eye 104 may reflect the infrared light that is incident thereupon. Such reflected infrared light may be received and measured by the imaging sensor 132. The imaging sensor 132 may be any imaging device operable to measure infrared light, including, but not limited to, charge-couple devices and active-pixel sensors, including CMOS and N-type MOS devices. The imaging sensor 132 may be positioned to receive the reflected infrared light either directly from the patient eye 104 or indirectly, such as where the infrared light reflected from eye patient eye 104 is incident upon and reflected by the hot mirror 138 in the direction of the imaging sensor 132. The reflected infrared light measured by the imagine sensor 132 may be analyzed to perform oculometry of the patient eye 104 responsive to what the patient eye observes on the retroreflective screen 120, namely, the stimuli or images produced by the projection device 110 and projected onto the retroreflective screen 120. The included eye imagery component may further comprise one or both of a fixed focus lens 135 and a focus variable lens 133 positioned optically intermediate the dichroic hot mirror 138 and the image sensor 132 to focus the reflected infrared light passing therethrough to be more clearly measurable by the image sensor 132.

The dichroic reflective and/or electrooptic device 136 may be configured to selectively permit light within a first wavelength range to pass therethrough and light within a second wavelength range to be reflected thereby. In the present embodiment, the dichroic reflective and/or electrooptic device 136 may be configured to electrooptically permit visible light, i.e. light having a peak intensity within a wavelength range from 380 nm to 750 nm, to pass therethrough. Accordingly, reflected light 106 from the retroreflective screen 120 may pass through the dichroic reflective and/or electrooptic device 136 and be incident upon and observable by the patient eye 104. In some embodiments, the first wavelength range within which light may pass through the dichroic reflective and/or electrooptic device may be narrower than all visible light. In some embodiments, the first wavelength range may be similar or equal to the wavelength range of light emitted by the display device 112. In some embodiments, the first wavelength range may be from 495 nm to 570 nm. In some other embodiments, first wavelength range may be electrooptically controlled to be either absorbed or scattered, thereby rendering the light path of the first wavelength range to be non-transparent to the patient eye. The second wavelength range may be configured to reflect light emitted by the infrared light source 134 and light reflected by the patient 104, specifically infrared light reflected by the patient eye 104. Accordingly, the second wavelength range may be from 700 nm to 1,000 nm.

Embodiments of the electrooptic device 136 may be any material or device operable to effectuate the above reflection/transmission/absorption/scattering, including, but not limited to, dichroic reflectors, switchable privacy smart film/glass including polymer dispersed liquid crystal and suspended particle embodiments, and electrochromic film/glass. Where electrochromic film/glass is used, the goggle device 102 may further comprise componentry necessary to control the state of the electrochromic film/glass. Where switchable privacy smart film/glass is used, the goggle device 102 may further comprise componentry necessary to control the state of the privacy switchable smart film/glass.

In the present embodiment, the combination of the display device 112 and the optical lens 114 may be such that light projected thereby has a projection accuracy on the retroreflective screen 120 within at least one of a range from −1.0° to 1.0°, a range from −0.5° to 0.5°, or a range from −0.25° to 0.25°.

The retroreflective screen 120 may be configured to retro-reflect light projected thereon, i.e. reflects light back in the direction of the light source. The retroreflective screen 120 may comprise any retroreflective material as is known in the art, including, but not limited to, fabric, laminate, film, paint, and combinations thereof. In some embodiments, the retroreflective screen 120 may comprise a flexible substrate with a retroreflective layer of material applied thereto, facilitating storage of the retroreflective screen 120.

The retroreflective screen 120 may be positioned a distance d from the goggle device 102 within the field of view 115. The distance d may be within a range from 1 meter to 3 meters. In one embodiment, the distance d is 2 meters. Distance d may be selected as a function of various parameters of the display device 112, including but not limited to, projection accuracy of light projected onto the retroreflective screen 120, angular resolution of light retro-reflected by the retroreflective screen 120, field of view of the display device 112, and the intensity of light emitted by the display device 112.

In some embodiments, the retroreflective screen 120 may be configured such that light reflected thereby has an angular resolution of at least one of less than or equal to 1.0°, less than or equal to 0.5°, or less than or equal to 0.25°.

Referring now to FIG. 4, additional details regarding the goggle device 102 are presented. The goggle device 102 may comprise a housing 103 configured to at least one of contain and permit attachment so as to carry the componentry of the projection device 110 and those components for performing eye imagery as described above. Moreover, the housing 103 may be configured to generally conform to the shape of a human head, such that patients having heads of various sizes and shapes can wear the goggle device 102. The dichroic reflective and/or electrooptic device 136 may be positioned in front of where the patient eye will be when the goggle device 102 is worn by the patient. The image sensor and the infrared light source may be positioned at a location in the housing 103 adjacent to an aperture 105 of the housing 103 through which electromagnetic radiation (EMR) may pass through. One or more of the optics mentioned above, including one or more of the optical lens 114, the focus variable lens 133, and the fixed focus lens 135, may be positioned in optical communication with the aperture 105, and further may be carried by an optic housing 107 attached to the housing 103. Moreover, the aperture 105 may be located at a location that is not observable by the patient eye, such that EMR passing through the aperture 105 is not directly observable by the patient.

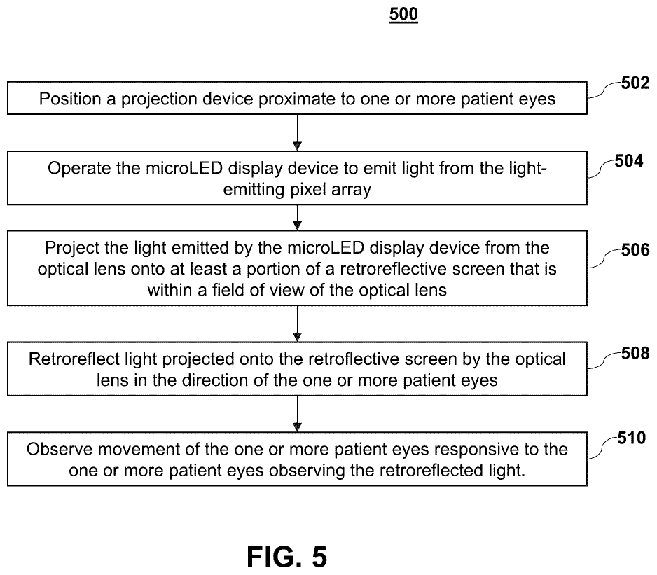

Referring now to FIG. 5, a method 500 according to an embodiment of the invention is presented. The method 500 may be with positioning a projection device proximate to one or more patient eyes at step 502. The projection device may comprise a microLED display device comprising a light-emitting pixel array and an optical lens positioned in optical communication with the microLED display device. The method 500 may continue at step 504 with operating the microLED display device to emit light from the light-emitting pixel array. The method 500 may continue at step 506 with projecting the light emitted by the microLED display device from the optical lens onto at least a portion of a retroreflective screen that is within a field of view of the optical lens. At step 508, the method 500 continues with retroreflecting light projected onto the retroflective screen by the optical lens in the direction of the one or more patient eyes. The method 500 may conclude at step 510 with observing movement of the one or more patient eyes responsive to the one or more patient eyes observing the retroreflected light.

The light emitted by the microLED display device may be a monochromatic green light. Operating the microLED display device to emit light from the light-emitting pixel array may include operating the microLED device to emit light from at least one of a pixel of the pixel array or a group of adjacent pixels of the pixel array such that an angular resolution of the emitted light on the retroflective screen is less than or equal to 0.5°. Operating the microLED display device to emit light from the light-emitting pixel array may include emitting light from at least one of a pixel of the pixel array or a group of adjacent pixels of the pixel array such that the projection of the emitted light has a projection accuracy on the retroflective screen within a range from −0.5° to 0.5°. Positioning the projection device proximate to one or more patient eyes may comprise positioning goggles comprising the projection device on a patient head. Operating the microLED display device to emit light from the light-emitting pixel array may include emitting light such that a two-dimensional motion simulation is projected onto the retroreflective screen.

As mentioned above, the projection device 110 may be positioned generally between where patient eyes are intended to be positioned. In the present embodiment, the projection device 110 may be positioned on a bridge section 109 of the housing 103 that may sit above the bridge of a patient's nose when the goggle device 102 is worn by the patient. Such a location is exemplary only and positioning the projection device 110 anywhere on the housing 103 is contemplated and included within the scope of the invention.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

1. An oculometry apparatus comprising:
a goggle device comprising a projection device, the projection device comprising:
a microLED display device comprising a light-emitting pixel array; and
an optical lens positioned in optical communication with the microLED display device and configured to project light within a field of view of the optical lens; and
a retroreflective screen positioned at a distance from the goggle device and configured to be positioned to overlap the field of view of the optical lens;
wherein the projection device is configured to be positioned proximate to one or more patient eyes; and
wherein the retroreflective screen is configured to retro-reflect light in the direction of the one or more patient eyes.

2. The oculometry apparatus of claim 1 wherein the microLED display device is monochromatic.

3. The oculometry apparatus of claim 2 wherein the microLED device is configured to emit green light.

4. The oculometry apparatus of claim 1 wherein light emitted from at least one of a pixel of the light-emitting pixel array or a group of adjacent pixels of the light-emitting pixel array and retro-reflected by the retroreflective screen has an angular resolution on the retroreflective screen of less than or equal to 0.5°.

5. The oculometry apparatus of claim 1 wherein light emitted from at least one of a pixel of the pixel array or a group of adjacent pixels of the pixel array has a projection accuracy on the retroreflective screen within a range from −0.5° to 0.5°.

6. The oculometry apparatus of claim 1 wherein the pixel array has dimensions of at least 120 horizontal pixels by 100 vertical pixels.

7. The oculometry apparatus of claim 1 wherein the field of view of the optical lens is greater than or equal to 60° horizontally and greater than or equal to 50° vertically.

8. The oculometry apparatus of claim 1 wherein the retroreflective screen comprises at least one of a retroreflective fabric, a retroreflective laminate, a retroreflective film, and a retroreflective paint.

9. The oculometry apparatus of claim 1 wherein the retroreflective screen is positioned at a distance within a range from 1 meter to 3 meters from the goggle device.

10. The oculometry apparatus of claim 1 wherein the projection device is positioned between two ocular apertures of the goggle device and emits light in the direction of a field of view of the patient when wearing the goggle device.

11. The oculometry apparatus of claim 1 further comprising:
an infrared light source;
an image sensor; and
a dichroic reflective device configured to permit transmission of visible light and reflection of infrared light and positioned in optical communication of each of the infrared light source, the image sensor, and an eye of the patient.

12. The oculometry apparatus of claim 1 wherein the microLED display device is operable to emit light such that a two-dimensional motion simulation is projected onto the retroreflective screen.

13. A method of performing an oculometric examination comprising:
positioning a goggle device comprising a projection device proximate to one or more patient eyes, the projection device comprising:
a microLED display device comprising a light-emitting pixel array; and
an optical lens positioned in optical communication with the microLED display device;
operating the microLED display device to emit light from the light-emitting pixel array;
projecting the light emitted by the microLED display device from the optical lens onto at least a portion of a retroreflective screen that is positioned at a distance from the goggle device within a field of view of the optical lens;
retroreflecting light projected onto the retroreflective screen by the optical lens in the direction of the one or more patient eyes; and
observing movement of the one or more patient eyes responsive to the one or more patient eyes observing the retroreflected light.

14. The method of claim 13 wherein the light emitted by the microLED display device is a monochromatic green light.

15. The method of claim 13 wherein operating the microLED display device to emit light from the light-emitting pixel array comprises operating the microLED device to emit light from at least one of a pixel of the pixel array or a group of adjacent pixels of the pixel array such that an angular resolution of the emitted light on the retroreflective screen is less than or equal to 0.5°.

16. The method of claim 13 wherein operating the microLED display device to emit light from the light-emitting pixel array comprises emitting light from at least one of a pixel of the pixel array or a group of adjacent pixels of the pixel array such that the projection of the emitted light has a projection accuracy on the retroreflective screen within a range from −0.5° to 0.5°.

17. The method of claim 13 the retroreflective screen is positioned at a distance within a range from 1 meter to 3 meters from the goggle device.

18. The method of claim 13 wherein operating the microLED display device to emit light from the light-emitting pixel array comprises emitting light such that a two-dimensional motion simulation is projected onto the retroreflective screen.

19. An oculometry apparatus comprising:
a goggle device configured to be worn on a patient head, comprising
two ocular apertures;
a projection device positioned between the ocular apertures and comprising:
a monochromatic microLED display device comprising a light-emitting pixel array the pixel array having dimensions of at least 120 horizontal pixels by 100 vertical pixels; and
an optical lens positioned in optical communication with the microLED display device and configured to project light within a field of view of the optical lens, the field of view being greater than or equal to 60° horizontally and greater than or equal to 50° vertically;
an infrared light source;
an image sensor; and
a dichroic reflective device configured to permit transmission of visible light and reflection of infrared light and positioned in optical communication of each of the infrared light source, the image sensor, and one or more patient eyes; and
a retroreflective screen configured to be positioned to overlap the field of view of the optical lens at a distance from the goggle device and comprising at least one of a retroreflective fabric, a retroreflective laminate, a retroreflective film, and a retroreflective paint;
wherein the retroreflective screen is configured to retro-reflect light in the direction of the one or more patient eyes; and
wherein light emitted from at least one of a pixel of the light-emitting pixel array or a group of adjacent pixels of the light-emitting pixel array and retro-reflected by the retroreflective screen has an angular resolution on the retroreflective screen of less than or equal to 0.5°.

20. The oculometry apparatus of claim 19 wherein light emitted from at least one of a pixel of the pixel array or a group of adjacent pixels of the pixel array has a projection accuracy on the retroreflective screen within a range from −0.5° to 0.5°.
